(19) United States
(12) Patent Application Publication
(10) Pub. No.: US 2021/0236235 A1
(43) Pub. Date: Aug. 5, 2021

(54) APPARATUS FOR DRYING AND SANITIZING REUSABLE TUBULAR MEDICAL DEVICES

(71) Applicants: Dennis Werger, Wells, ME (US); Robert Beebe, Raleigh, NC (US); David Allan Marsh, Marina Del Rey, CA (US); Bruce Lampert, Newton, MA (US)

(72) Inventors: Dennis Werger, Wells, ME (US); Robert Beebe, Raleigh, NC (US); David Allan Marsh, Marina Del Rey, CA (US); Bruce Lampert, Newton, MA (US)

(21) Appl. No.: 16/948,751

(22) Filed: Sep. 30, 2020

Related U.S. Application Data
(60) Provisional application No. 62/885,632, filed on Aug. 12, 2019.

(51) Int. Cl. A61B 90/70

(52) U.S. Cl. CPC A61B 90/70; A61B 2090/701

(57) ABSTRACT

An apparatus for drying and sanitizing reusable tubular medical devices, wherein the apparatus is embodied in a method that includes inserting the apparatus in the reusable tubular medical device, such as endoscopic tubing, thereby removing moisture, killing bacteria, and preventing bacteria growth.

### CROSS-REFERENCE TO RELATED APPLICATION

This application claims the benefit of priority of U.S. provisional application No. 62/885,632, filed 12-AUG.-2019, the contents of which are herein incorporated by reference.

### BACKGROUND OF THE INVENTION

The present invention relates to tube cleaning devices, systems and methods and, more particularly, an apparatus for drying and sanitizing reusable tubular medical devices, wherein the apparatus may be inserted into the reusable tubular medical device, such as endoscopic tubing, thereby removing moisture, killing bacteria, and preventing bacteria growth.

Endoscopes and other reusable tubular medical and lab equipment have channels that are difficult to clean and/or completely dry between uses. Any remaining bacteria presents a health risk during subsequent use of the device. Furthermore, even small traces of bacteria can exponentially mushroom into a bigger risk. Any residual moisture in the tubular medical device affords a breeding ground for bacteria. Multiple studies of post-cleaned endoscopes have shown that air drying of channels is time consuming and ineffective in eliminating the bacteria and moisture that supports bacteria. Further published studies have reported that manual drying, as currently practiced, does not completely dry the internal channels. Also, human error and motivation to rush to reuse the device as quickly as possible contributes to incomplete drying. In short, the reusable design and current cleaning process of flexible endoscopes and other medical tubing puts patients at risk for cross-contamination.

In the case of an endoscope, for example, the current standard practice after cleaning is hanging them on a custom rack for a defined period of time to allow moisture to drip out of the channel and eventually dry. Alternatively, the scope is placed in an expensive custom drying machine that circulates air around the scope and through the channel to complete the drying cycle in less time than manual drying. While both methods are generally effective for drying the channel, they are slow and expensive, and not totally effective in removing bacteria. Moreover, endoscope dryers and other mechanical customized endoscope drying systems often require a separate room or added space to set up and use the equipment.

The endoscope is a complex piece of mechanical medical equipment which provides a great tool for physicians to examine and treat patients. This complexity leaves many segments of the endoscope with small and difficult to reach segments that require cleaning between procedures. While conventional cleaning of the endoscope, when done properly, is effective in removing most of the procedure-induced bacteria, the high-stakes of human health combined with the complex design factors and the inevitable likelihood of human error can result in a significant source of residual bacteria and moisture that can harm downstream patients. Studies have also shown that retraining of the cleaning staff only slightly improves the results. The present invention is a simple tool embodied in a simple process that would significantly reduce the risk of an endoscope being used on a patient with residual bacteria and/or moisture in the endoscopic channels.

As can be seen, there is a need for apparatus for drying and sanitizing tubular medical devices, wherein the apparatus may be inserted into the lumen of the reusable tubular medical device, such as an endoscopic tubing, to kill bacteria or prevent bacteria growth and/or remove moisture.

The present invention provides an additional tool to help ensure that residual moisture in the channels or tubing is absorbed into the outer fiber of the present invention and that any bacteria in the channel or in the moisture is killed by the antimicrobial component of the apparatus. The present invention is unique as there are no devices that can kill bacteria and remove moisture from the working channels or elevators of endoscopes or other reusable tubular medical devices.

### SUMMARY OF THE INVENTION

In one aspect of the present invention, an apparatus for drying and sanitizing a tubular medical device, the apparatus including the following: a core extends axially in relation to the tubular medical device between a proximal end and a distal end, thereby defining a core length; a fibrous mass sheathed along a substantial portion of the core length; the fibrous mass having a continuous semi-circumferential spheroidic profile, wherein the continuous semi-circumferential profile has a sufficient radial dimension so that a portion of the continuous semi-circumferential profile engages a wall defining a lumen of the tubular medical device; and the fibrous mass comprising an absorbent property and an antimicrobial property.

In another aspect of the present invention, apparatus for drying and sanitizing a tubular medical device, the apparatus including the following: a non-rigid, semi-rigid or flexible core extends axially in relation to the tubular medical device between a proximal end and a distal end, thereby defining a core length; a fibrous mass sheathed along a substantial portion of the core length; the fibrous mass having a continuous semi-circumferential profile, wherein the continuous semi-circumferential profile has a sufficient radial dimension so that a portion of the continuous semi-circumferential profile engages a wall defining a lumen of the tubular medical device; the fibrous mass comprising an absorbent property and an antimicrobial property; and an atraumatic tip protruding from a distal end of the fibrous mass.

In yet another aspect of the present invention, method of cleaning a tubular medical device, the method including the following: urging a fibrous mass in a lumen of the tubular medical device in an engaged condition, wherein the fibrous mass is sheathed a non-rigid, semi-rigid or flexible core, wherein the fibrous mass having a continuous semi-circumferential profile, wherein the continuous semi-circumferential profile has a sufficient radial dimension so that a portion of the continuous semi-circumferential profile engages a wall defining said lumen in the engaged condition, and wherein the fibrous mass comprising an absorbent property and an antimicrobial property; and maintaining the fibrous mass in the engaged condition for a predefined time period, the predefined time period a function of the antimicrobial property.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, showing the non-rigid, semi-rigid or flexible core extending out from a distal end of a fibrous mass;

FIG. 2 is a perspective view of an exemplary embodiment of the present invention, showing a first atraumatic tip extending out from a distal end of a fibrous mass;

FIG. 3 is a perspective view of an exemplary embodiment of the present invention, showing a first atraumatic tip extending out from a distal end of a fibrous mass;

FIG. 4 is a perspective view of an exemplary embodiment of the present invention, showing no extended tip extending out from a distal end of a fibrous mass;

FIG. 5a is an elevation view of an exemplary embodiment of the present invention, demonstrating an insertion of the present invention into an endoscopic tube;

FIG. 5b is an elevation view of an exemplary embodiment of the present invention, demonstrating the sanitization of the endoscopic tube; and

FIG. 5c is an elevation view of an exemplary embodiment of the present invention, demonstrating removal of the present invention from the endoscopic tube.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

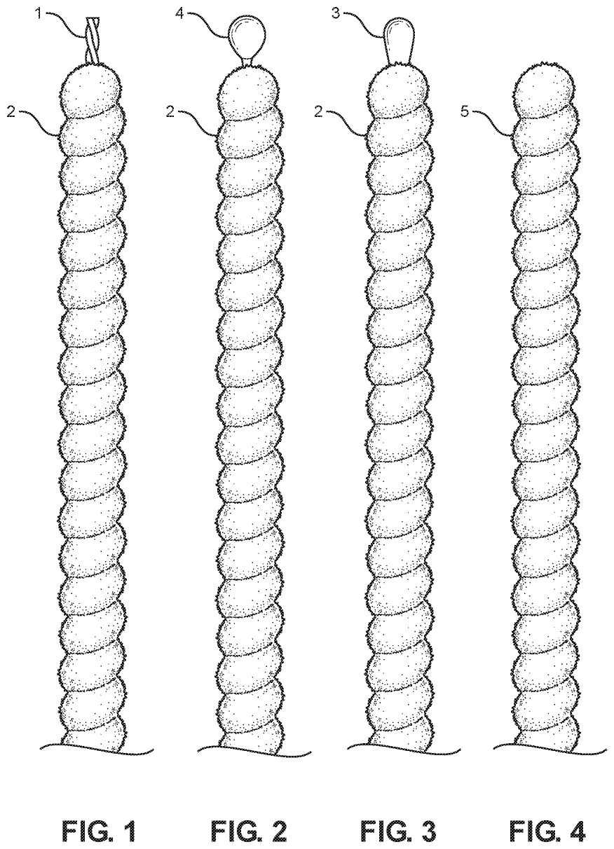

Referring now to FIGS. 1 through 5c, the present invention may include an apparatus 10 having a non-rigid, semi-rigid or flexible core 1 extending between a proximal end a distal end. The core 1 could be various plasticized materials or metallic material. The core 1 could be a braided element such as strands of wire braided or twisted together. Non-rigid, semi-rigid or flexible core provides desired axial rigidity while resisting bowing deformation due to axial compressive forces; semi-rigid may be non-rigid, flexible or at least providing sufficient rigidity to prevent bowing deformation of greater than three-quarters of an inch with a load of contemplated by the disclosure herein.

Integral to the core 1 may be a mass of fiber or fibrous mass 2 that can be formed from any number of common or unique materials such as polyester, rayon, cotton, microfiber, blend, etc. The fibrous mass 2 may have a continuous semi-circumferential profile illustrated in the FIGS. By continuous semi-circumferential spheroidic profile, it should be understood that the fibrous mass 2 forms a series of abutting less-than fully formed spheroid shapes, wherein the spheroid shapes can be prolate spheroid, oblate spheroid, and in certain embodiments an apparent combination of both prolate and oblate spheroids in shape. ‘Less than fully formed,’ is understood to be that each spheroid shape may be truncated, or in other words the distance from center of the spheroid along the symmetry axis is cut/terminated prior to the symmetry axis reaching the would-be pole (the symmetry axis could be the axial axis of the core). In certain embodiments, the spheroid shapes could be truncated spherical shapes, even though the Figures show just the truncated, spheroid shapes. Likewise, the fibrous mass 2 could be defined by non-truncated spheroid (or spherical) shapes.

In certain embodiments, the fibrous mass 2 has inherent absorbent properties or as a result of additives to the fiber. The fiber may be attached to the core 1 mechanically, by way of adhesives, melting, or any suitable method. The fiber may be connected along the working length of the core 1. The fiber is antimicrobial by material selection or by way of additive treatment.

A tip protector 3 or 4 of atraumatic material or design may cover a distal portion of the core 1 protruding out from a distal end of the fibrous mass 2. A first tip protector 3 may be volumetrically oblong, while a second tip protector 4 may be generally spherical, as illustrated in FIGS. 3 and 2, respectively. In other embodiments, the core 1 protrudes out of the distal end of the fibrous mass 2, as illustrated in FIG. 1. While in a non-protruding embodiment 5, the core 1 does not protrude at all from the distal end of the fibrous mass 2, as illustrated in FIG. 4.

In some embodiments, an independent non-protruding embodiment 5 of generally the same design may be specifically designed and sized to be placed behind the elevator segment of a duodenoscope or endoscopic ultrasound scope, or similar designed scopes, that performs the same function as the primary invention.

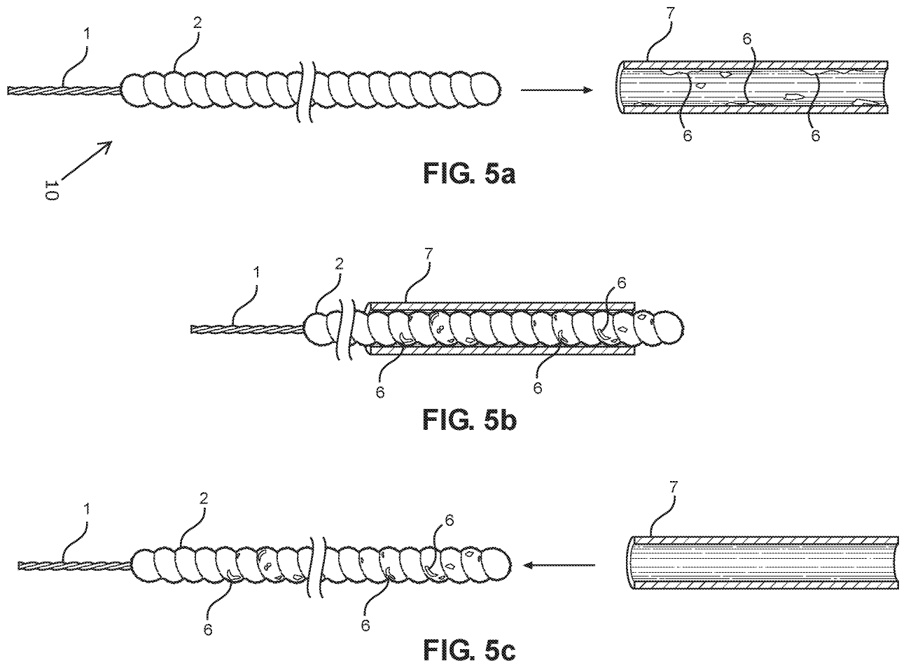

Referring to FIGS. 5a-5c, the solid or braided non-rigid, semi-rigid or flexible core 1 with the antimicrobial and/or absorbent fibrous mass 2 attached and the distal atraumatic tip 3 or 4 are inserted down the length of the internal lumen of a tubular medical device 7 to be cleaned or dried. The fibers 2 contact the internal walls of the said tubular medical device 7 and absorb moisture 6 and contact and kill any bacteria along the internal lumen of said tubular medical device 7. The apparatus 10 is inserted until its distal end of the fibrous mass 2 exits the distal end of said tubular medical device 7, as illustrated in FIG. 5b. The present invention may be left in place for a predefined time period which may be dictated by the antimicrobial and/or drying intention of the tubular medical device 7 that the invention is inserted into.

Another iteration of the invention may be using a short segment of the invention, with or without the atraumatic tip 3 or 4, to assist in killing bacteria and/or drying behind the elevator segment, for example, that is present if the tubular medical device 7 is an endoscope. Similar to other embodiments, said shorter segment of the present invention may be composed of a core material that is non-rigid, semi-rigid or flexible with antimicrobial and/or absorbent fibers attached. It may have atraumatic tip segments, depending on the clinical application and risk of damage to the elevator segment of the endoscope. The present invention would be inserted behind the elevator and left in place for a designated time period to complete the intended objective of absorbing any residual post cleaning moisture and/or killing of any residual bacteria.

A method of manufacturing the present invention may include the following. Fiber of cotton, polyester, rayon, polypropylene, microfiber or any textile material blend may be treated with the antimicrobial material before or after assembly to the core material. If the core material is a twisted or braided wire, then the fiber may be incorporated into the twisting or braiding process of the core. If the fiber is attached to a plastic or similar core material via adhesive or molding or melting the antimicrobial component or additional absorbent material may be added before or after attachment. The antimicrobial and/or absorbent components may be added during the production of the fiber material as well. These components can be blended into the fiber material or added to the fibrous mass 2 in a coating process appropriate for the fiber material, such as dipping, spraying or other mechanical means.

The atraumatic tip protectors may be added or formed when the present invention is cut to working lengths. These tips can be made of any material where the shape can be formed to eliminate sharp or pointed ends. The tips could be bent, formed, or have a welded shape or be attached by adhesive, melting, molding, etc.

The outside dimension, length, stiffness, absorbency and density of the fiber and core can be modified as necessary per the specific endoscope or channel or tubing being treated by the invention. Any type of fiber material may be utilized as long as it is absorbent and antimicrobial or can be treated or modified to be absorbent and antimicrobial.

In a first iteration of the invention the antimicrobial material is projected to be ionic silver, ionic copper, or ionic zinc or any combination thereof. Future variants of the invention may include other antimicrobial or antibiotic materials such as chlorohexidine, PHMB (polyhexamethylene Biguanide), Quaternary ammonium cations (Quats) or any number of materials or chemicals that would have an effective bacterial kill rate alone or in combination. The atraumatic tip can be either formed from the distal end of the non-rigid, semi-rigid or flexible core tip or attached to the distal end tip of the non-rigid, semi-rigid or flexible core.

The non-rigid, semi-rigid or flexible core is adapted to allow for pushing the present invention down the length of the endoscope channel or lumen of the tubular medical device 7 without undue bowing deformation. The fibers may be adapted to attach to the non-rigid, semi-rigid or flexible core and where the fibrous mass is absorbent or treated to be absorbent and incorporate an antimicrobial material or are at least partially coated with an antimicrobial material.

The length of the apparatus 10 can be varied during production to effectively be used on the intended device length. Fibers that are attached to the core may be modified or cut during production to properly match the internal lumen of the device to allow for internal lumen contact and still allow for advancement down the length of the device channel.

A cleaning process of an endoscope or other tubular medical device 7 may include a properly sized version of the apparatus 10 being removed from its packaging and inserted into the proximal end of the channel or tubular medical device 7. The present invention may be fed through the entire length of the channel or lumen of the tubular medical device 7 until it protrudes from the distal end of the channel or lumen of the tubular medical device 7.

The present invention is then left in place for a minimum designated period of time base the application's need. To remove the present invention the distal end of the invention that protrudes from the distal end of the channel or tubing is grasped and slowly pulled until the proximal end of the invention exits the channel or lumen of the tubular medical device 7. The present invention may then be discarded.

Another iteration of the present invention may be a short segment that is essentially the same length as the channel or lumen of the tubular medical device 7—it has antimicrobial and/or absorbency properties and may be larger in outside diameter and may not have atraumatic tips on either end. This iteration of the present invention may be removed from the package and the distal end is inserted behind the elevator segment of an endoscope. It is fully inserted until the distal end of the present invention contacts the bottom of the elevator segment. The present invention may be slightly rotated to help insure complete contact with the area behind the elevator. The present invention may be left in place for a minimum period of time depending on the intended outcome.

Both iterations of the invention may be left in place for multiple days as long as the antimicrobial material is sufficiently effective in killing bacteria. Once the endoscope or tubular medical device 7 is needed for a procedure any part of the present invention that is still in the device or tubing would be removed.

Additionally, the present invention can be used in any reusable medical, lab, or foodservice equipment that has channels that need to be dried and protected from bacteria between use or while in storage awaiting use.

It should be understood, of course, that the foregoing relates to exemplary embodiments of the invention and that modifications may be made without departing from the spirit and scope of the invention as set forth in the following claims.

What is claimed is:

1. An apparatus for drying and sanitizing a tubular medical device, the apparatus comprising:
a core extends axially in relation to the tubular medical device between a proximal end and a distal end, thereby defining a core length;
a fibrous mass sheathed along a substantial portion of the core length;
the fibrous mass having a continuous semi-circumferential profile, wherein the continuous semi-circumferential spheroidic profile has a sufficient radial dimension so that a portion of the continuous semi-circumferential spheroidic profile engages a wall defining a lumen of the tubular medical device; and
the fibrous mass comprising an absorbent property and an antimicrobial property.

2. The apparatus of claim 1, wherein the absorbent property is added to the fibrous mass.

3. The apparatus of claim 1, wherein the antimicrobial property is added to the fibrous mass.

4. The apparatus of claim 1, wherein the core is semi-rigid.

5. The apparatus of claim 1, wherein the distal end of the core protrudes from a distal end of the fibrous mass.

6. The apparatus of claim 1, further comprising an atraumatic tip protruding from a distal end of the fibrous mass.

7. The apparatus of claim 6, wherein the atraumatic tip is spheroid.

8. The apparatus of claim 6, wherein the atraumatic tip is volumetrically oblong.

9. An apparatus for drying and sanitizing a tubular medical device, the apparatus comprising:
a semi-rigid core extends axially in relation to the tubular medical device between a proximal end and a distal end, thereby defining a core length;
a fibrous mass sheathed along a substantial portion of the core length;
the fibrous mass having a continuous semi-circumferential spheroidic profile, wherein the continuous semi-circumferential profile has a sufficient radial dimension so that a portion of the continuous semi-circumferential spheroidic profile engages a wall defining a lumen of the tubular medical device;
the fibrous mass comprising an absorbent property and an antimicrobial property; and
an atraumatic tip protruding from a distal end of the fibrous mass.

10. A method of cleaning a tubular medical device, the method comprising:
urging a fibrous mass in a lumen of the tubular medical device in an engaged condition, wherein the fibrous mass is sheathed a semi-rigid core, wherein the fibrous mass having a continuous semi-circumferential spheroidic profile, wherein the continuous semi-circumferential profile has a sufficient radial dimension so that a portion of the continuous semi-circumferential spheroidic profile engages a wall defining said lumen in the engaged condition, and wherein the fibrous mass comprising an absorbent property and an antimicrobial property; and
maintaining the fibrous mass in the engaged condition for a predefined time period, the predefined time period a function of the antimicrobial property.
